(19) United States
(12) Patent Application Publication
(10) Pub. No.: US 2004/0116799 A1
(43) Pub. Date: Jun. 17, 2004

(54) Compatibility of accessory to magnetic resonance

(76) Inventor: Ravi Srinivasan, Beachwood, OH (US)

Correspondence Address: Mark D. Saralino, Renner, Otto, Boisselle & Sklar, LLP, 1621 Eucid Avenue, Nineteenth Floor, Cleveland, OH, 44115-2191, US

(21) Appl. No.: 10/723,325

(22) Filed: Nov. 26, 2003

Related U.S. Application Data
(60) Provisional application No. 60/429,911, filed on Nov. 29, 2002.

(51) Int. Cl. A61B 5/055; G01V 3/00

(52) U.S. Cl. 600/410; 324/318; 324/322

(57) ABSTRACT

An incubator for use with a magnetic resonance (MR) system, the incubator minimizing an amount of interference generated during an MR scan of the incubator is disclosed. The incubator includes an incubator housing, which includes a patient compartment, an aggregate compartment coupled to the patient compartment, and an electronics compartment coupled to the aggregate compartment. The incubator housing and an incubator frame are constructed as a uni-body assembly. A means for inhibiting insertion of the electronics compartment of the incubator housing into the MR system also is provided. Additionally, a method for improving the compatibility of a magnetic resonance (MR) accessory for maintaining or monitoring the health of a patient while undergoing magnetic resonance imaging (MRI) with an MR system is disclosed. The method includes at least one of the steps of reducing an interference between the accessory and a static magnetic field of the MR system, reducing an interference between the accessory and a time varying gradient magnetic field of the MR system, reducing radio frequency (RF) interference between the accessory and the MR system; and reducing electromagnetic interference (EMI) between the accessory and the MR system..

### CROSS REFERENCE TO RELATED APPLICATIONS

This Application claims priority from U.S. Provisional Application Serial No. 60/429,911 filed Nov. 29, 2002, which is incorporated herein by reference.

### TECHNICAL FIELD

The present invention relates to a magnetic resonance (MR) system. Specifically, the invention relates to improving the compatibility of accessories to MR systems. More specifically, the invention relates to improving the MR compatibility of an incubator system for use in MR systems.

### BACKGROUND

NMR or MRI

In Magnetic Resonance Imaging (MRI) systems and Nuclear Magnetic Resonance (NMR) systems, a static magnetic field (B) is applied to a body under investigation. The static magnetic field defines an equilibrium axis of magnetic alignment in a region of the body under investigation. A radio frequency (RF) field is applied in the region being examined in a direction orthogonal to the static field direction. The RF field excites magnetic resonance in the region, and resulting RF signals are detected and processed. Generally, the resulting RF signals are detected by RF coil arrangements placed close to the body. See for example, U.S. Pat. No. 4,411,270 to Damadian and U.S. Pat. No. 4,793,356 to Misic et al. Typically, these coils are either surface type or volume type coils, and, depending on the application, are used to transmit RF and receive NMR signals from the region of interest (ROI).

An incubator system for magnetic resonance (MR) was introduced by Lammers (see, e.g., European Patent Application No. EP 1 249 216 A1 and PCT Patent Application No. WO 02-083053 A1). The incubator system allows sick patients to be transported from the neonatal intensive care unit (NICU) to the MR system for effective diagnosis/prognosis by MR. The environment within this incubator is similar to the incubators commonly found in the NICU (e.g., air temperatures up to 39 degrees C., high humidity up to 100% relative humidity and high levels of oxygen in the circulating air up to 100%). Another incubator system was introduced by Rohling et al. (see U.S. Pat. No. 6,611,702). The incubator system of Rohling et al. includes an incubator arrangement and radio frequency coil for use in a magnetic resonance imaging system. Additional information relating to incubator systems can be found in U.S. Pat. No. 5,800,335 issued to Koch et al, and PCT Patent Application No. WO 98/48756 to Nordell et al.

Diagnosis/prognosis of a patient within the incubator depends on the quality of the MR image that is obtained from the MR system in conjunction with the incubator. Additionally, for patient safety, it is necessary that the interference between the MR and the incubator be minimized. Likewise, the performance of the incubator functions must not be compromised while in the presence of MR. Further, for effective diagnosis/prognosis, it is necessary that the interference between the incubator and the MR be maintained at a very low level such that the diagnosis is not affected. This necessitates that the compatibility of the incubator with the MR system be improved such that patient safety is not compromised and that the two systems (incubator, MR) operate independently.

### SUMMARY OF THE INVENTION

An object of the invention is to improve the compatibility of an incubator system with an MR system. Another object of the invention is to incorporate filtering such that the incubator system is safe for use with different field strength MR systems and over a wide frequency range for a given magnetic field strength.

Three types of interferences must be dealt with when dealing with MR. These interferences are due to static magnetic fields, time varying magnetic fields and radio-frequency (RF) fields at the NMR frequency of operation. Interferences can be complicated when all three are present, which generally is the case with an MR scanner.

According to one aspect of the invention, the invention is directed to an incubator for use with a magnetic resonance (MR) system. The incubator minimizing an amount of interference generated during an MR scan of the incubator, and includes: an incubator housing, including a patient compartment, an aggregate compartment coupled to the patient compartment, and an electronics compartment coupled to the aggregate compartment; an incubator frame, wherein the incubator frame and the incubator housing are constructed as a uni-body assembly; and a means for inhibiting insertion of the electronics compartment of the incubator housing into the MR system.

Another aspect of the invention relates to a method for improving the compatibility of a magnetic resonance (MR) accessory for maintaining or monitoring the health of a patient while undergoing magnetic resonance imaging (MRI) with an MR system. The method includes at least one of the steps of: reducing an interference between the accessory and a static magnetic field of the MR system; reducing an interference between the accessory and a time varying gradient magnetic field of the MR system; reducing radio frequency (RF) interference between the accessory and the MR system; and reducing electro-magnetic interference (EMI) between the accessory and the MR system.

Other aspects, features, and advantages of the invention will become apparent from the following detailed description. It should be understood, however, that the detailed description and specific examples, while indicating several embodiments of the present invention, are given by way of illustration only and various modifications may naturally be performed without deviating from the present invention.

### BRIEF DESCRIPTION OF DRAWINGS

These and further features of the present invention will be apparent with reference to the following description and drawings, wherein:

FIG. 1 is a block diagram of an MR system that can be used in conjunction with the present invention;

FIG. 2 is an isometric view of an incubator in accordance with an embodiment of the present invention;

FIGS. 3-8 illustrate various filter networks and their corresponding frequency response plot in accordance with the present invention;

FIGS. 9-12 illustrate various filter networks for suppressing RF interference in accordance with the present invention.

### DISCLOSURE OF INVENTION

The following is a detailed description of the present invention with reference to the attached drawings, wherein like reference numerals will refer to like elements throughout.

The quality of an image obtained from an MR system is dependent on various factors, including, for example, interference with a static magnetic field of the MR system, interference from time varying gradient magnetic fields, RF interference and electro-magnetic interference (EMI). Accordingly, when obtaining MR images, it is desirable to reduce or minimize such interference.

When obtaining MR images of infants, it is desirable to keep the infant in an incubator during the MR scan. The incubator maintains a specified atmosphere around the infant that promotes the well-being of the infant. Unfortunately, when an MR scan is performed on an infant who is within an incubator, interference is generated due to the presence of the incubator. As a result, the image quality obtained from the MR system is degraded.

The present invention relates to a method of improving the compatibility of accessories, such as an incubator, a ventilator, a patient monitor, etc., with MR. The present invention will be described with respect to an incubator. It will be appreciated, however, that the present invention can be applied to other accessories for MR, and the discussion with respect to an incubator is merely exemplary and not intended to be limiting in any way. The present invention minimizes the interference between an accessory and an MR system when the accessory is placed within or near the MR system, thus increasing the quality of images obtained from the MR system.

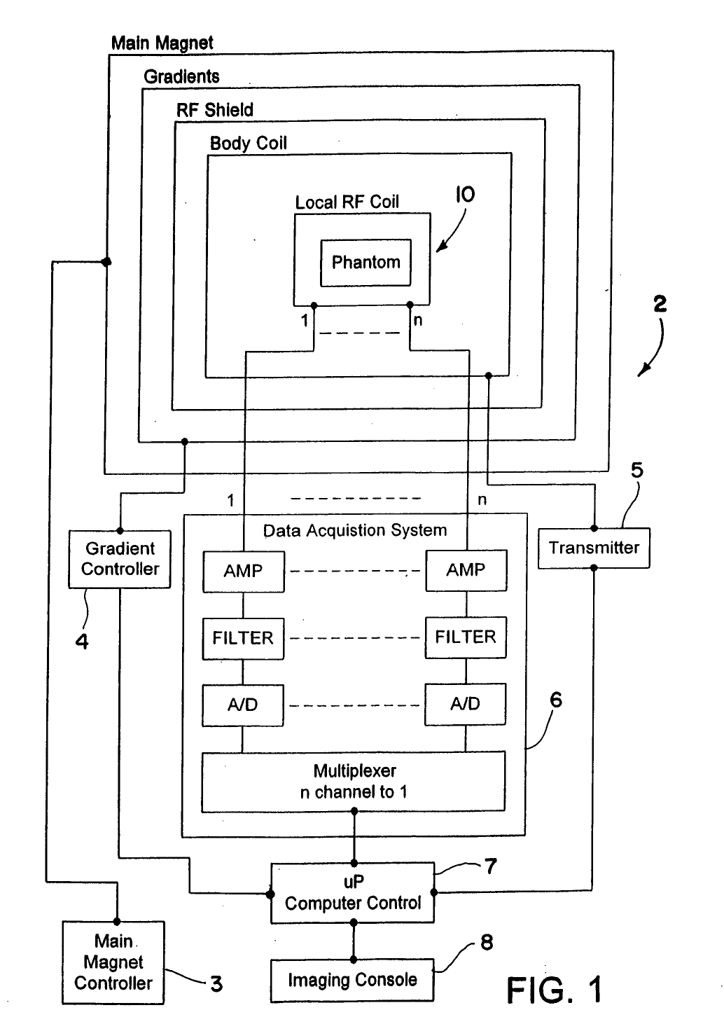

Referring to FIG. 1, a block diagram of an MR system 2 that may be used in conjunction with an incubator in accordance with the present invention is shown. The MR system 2 includes a main magnet controller 3, a gradient controller 4, a transmitter 5 and a data acquisition system 6, as is conventional. A computer controller 7 controls the operation of the system, and system data is provided to a user through an imaging console 8. The coil 10 sends and receives data to/from the data acquisition system 6.

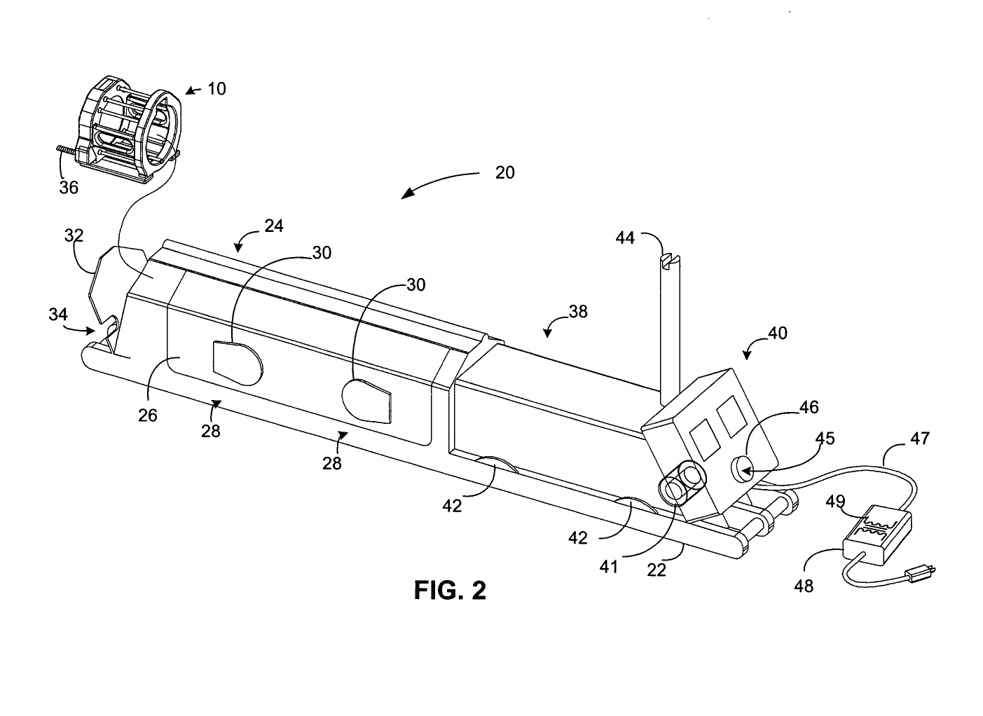

Referring to FIG. 2, an incubator/RF coil arrangement in accordance with an embodiment of the present invention is illustrated. An incubator 20, which is portable and can be lifted and transported by two people, includes an incubator base 22. The incubator base 22 is of a “uni-body” construction; that is, it is made of a single piece to provide a certain amount of rigidity to the incubator structure and to reduce image artifacts due to vibration inside the MR scanner. The incubator 20 includes three sections; patient, aggregate and electronics compartments.

The patient compartment 24 is the longest section of the three, and, for example, is made of a clear plastic material, thus permitting complete visual contact of a patient inside the incubator. The patient compartment 24 includes a patient bed (not shown) that quickly can be placed in or removed from the incubator 20, and a soft mattress (not shown), which is placed atop the patient bed. The patient compartment 24 also includes double-walled doors 26 on both sides of the incubator. The doors 26 can be swiveled up for immediate and complete patient access. The doors 26 include shaped access panels 28 with flap doors 30 (small, two shown) for limited access to the patient. A flap door 32 toward the rear of the incubator is used for quick introduction/removal of the RF coil 10 pre/post MR scan. A slot 34 in the rear flap door 32 allows a cable 36 from the RF coil 10 to be connected to the MR system (not shown). Likewise small openings are provided (not shown) in the patient section to allow intra-venous (fluid), life sustaining (oxygen through nasal canula or air/oxygen via a ventilator) and monitoring lines (vital signs such as, ecg (electro-cardio gram), saturated partial oxygen, n tidal carbon dioxide, blood pressure, etc.) to be coupled to the patient.

The aggregate compartment 38 is located between the patient compartment 24 and the electronics compartment 40. Air is warmed and humidity is generated in the aggregate compartment for circulation through the incubator. The aggregate compartment includes a fan blower 41, which helps draw fresh air through a particle filter (not shown) and circulate humidified warm air throughout the incubator as prescribed by a doctor (e.g., dialed in the electronics section by the user). A semi-circular guide 42 alongside the aggregate compartment 38 helps keep the lines, which are coupled to the patient, intact during transport and during the MR scan (and thereby minimizing the likelihood of the lines becoming pinched). An intra-venous (IV) pole 44 is attached to the aggregate compartment 38 and, in addition to serving as an IV stand, prevents the electronics compartment 40 from being inserted into a magnet bore (not shown) of the MR system.

The electronics compartment 40 includes the microprocessor control/feedback circuits that continuously monitor the incubator functions (e.g., air temperature, humidity, oxygen levels), and an operator interface, which includes safety alarms. The electronics compartment 40 also includes a motor 46 that is used to propel the fan blower 41 in the aggregate compartment 38. As was noted previously, the IV pole 44 prevents the electronics compartment 40 from being placed in the magnet bore and, thus, also prevents the motor 45 from being placed in the magnet bore. It should be appreciated that an electrical stop (not shown) can be used as an alternative to a mechanical stop (e.g., the IV pole). For example, an electrical switch can enable/disable power to the MR system depending on the orientation of the incubator relative to the MR system. If the orientation is improper, then power to the MR system is disabled.

As will be described in more detail below, the motor 45 is shielded with a metal cylinder 46 to minimize interference between the motor and the MR system. A power supply cable 47 is connected to the electronics compartment 40 and coupled to filtered main lines inside an MR room through a power supply box 48, which houses an isolation transformer 49. The remotely mounted and isolated transformer minimizes interference between the transformer and the MR system, thereby ensuring that power to the incubator will not be interrupted due to MR.

MR images of an infant are obtained by placing the incubator 20, including the infant, within the MR system 2. An MR scan of the infant is obtained using conventional techniques, and an image is produced. Since the incubator 20 produces minimal interference with the MR system 2 and a custom pediatric RF coil 10 is used (as described U.S. Patent Application filed by Ravi Srinivasan and titled Improved Radio Frequency Coil for Resonance Imaging Analysis of Pediatric Patients, filed concurrently herewith), image quality is improved. Moreover, the infant is not removed from the incubator 20 and thus the micro environment surrounding the infant is not disturbed.

As was noted above, there are several forms of interference that can be generated when an incubator is placed in an MR system and an MR scan is performed. Methods of reducing these forms of interference will now be discussed in more detail.

Static Magnetic Fields

Interference with static magnetic fields can be reduced or eliminated by using non-interference generating components, such as non-magnetic components and/or non-conductive, non-metallic plastic components. These types of components do not produce a water signal, and thus artifacts due to the components can be eliminated. For example, circulating currents within the components can be eliminated through the use of non-conductive materials.

Additionally, the components should be transparent to the main magnetic field of the MR system 2. Metal components should be non-magnetic (e.g., strontium, phosphor-bronze, beryllium-copper, copper, aluminum, silver, gold etc.) and preferably have a low permeability, e.g., a permeability that will cause less than 1 percent eddy currents, ghosting and/or distortion of the image in all three axis X, Y, Z, respectively, particularly in low signal to noise scans with echo times less than 2.0 milliseconds. In most cases, diamagnetic and ferro-magnetic materials must be limited, and in some cases diamagnetic and ferro-magentic materials should not be used.

The incubator 20 of the present invention is constructed from the above described materials, including plastics that are transparent to MR, are capable of operating at high temperatures, have low water absorption, and do not react with oxygen.

Electrical components within the incubator 20 are shielded to minimize interference with static magnetic fields. For example, the incubator of the present invention includes a magnetic fan motor 45. The motor 45 is shielded with a steel cylinder 46 and held to the incubator base 22. The steel cylinder has a thickness of about 1/16 inch, for example. Fasteners, such as steel screws and shafts (not shown), are replaced with beryllium-copper, phosphorous-bronze or aluminum, for example. Power is provided to the incubator via the incubator power supply 48, which is driven by the magnetic core transformer 49. The transformer 49 is mounted remotely from the incubator 20 to minimize any interference between the transformer and the MR system 2.

The performance of a remotely mounted transformer 49 with the incubator 20 in accordance with the present invention has been tested with an MR system 2. It was determined that the remotely mounted transformer is effective and does not starve the incubator power supply 48 during MR system operation (e.g., the remotely mounted transformer core does not become saturated from the strong magnetic fields of the MR system and, thus, the voltage delivered by the transformer is relatively constant), provided that the transformer is kept 5 feet or more away from a 1.6 meter long 1.5 Tesla (T) MR magnet.

Time Varying Gradient Magnetic Fields

Interference due to time varying gradient magnetic fields can be reduced using intermediate frequency (IF) filters. For example, IF filters and feed-thru capacitors can be placed in all signal lines (e.g., data carrying lines), wherein the feed-thru capacitors either block all of the interferences or shunt them to ground. Additionally, gradient interferences can be minimized by reducing the size of the metals used in shielding the incubator electronics or by keeping them away from the gradient field of view (FOV). Ghosting or aliasing can be minimized by eliminating moving metal parts and by placing the metal sections away from the gradient cross-overs along the magnet axis.

RF Interference

RF interference can be minimized by appropriate filtering mechanisms in passive signal lines and the active lines (lines that carry power). RF chokes can be used to prevent RF leakage, whereas high power RF filters capable of carrying a few amperes with very high impedances can be utilized. RF chokes and high power RF filters are made narrow-band or broad-band depending on the application. Broad-band attenuation is sought if the incubator system is to be used on different field strength MR systems and for cases where different frequencies are planned for a given MR field strength.

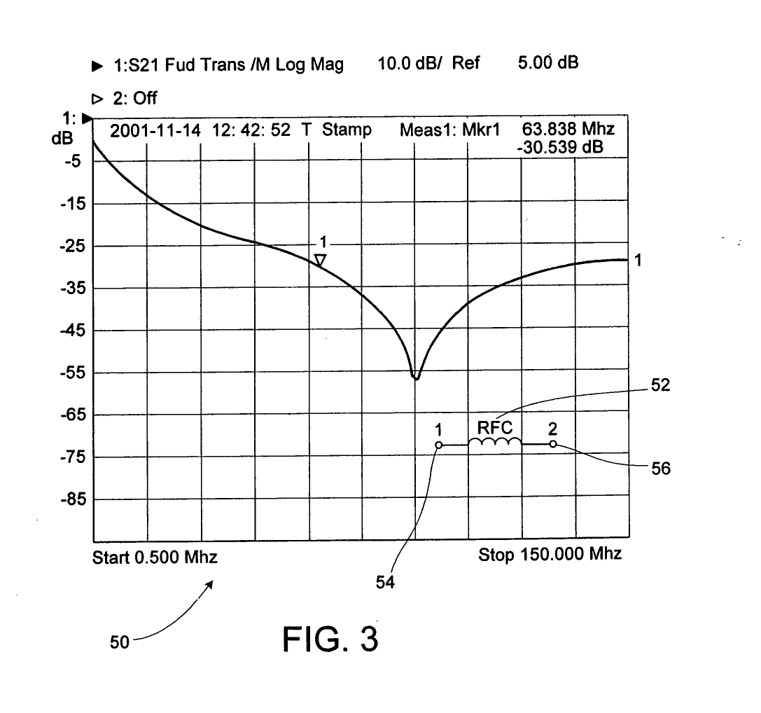

Referring to FIGS. 3-8, six schemes of filtering passive or active lines are included using RF chokes, tuning capacitors and high value RF shorting capacitors along with experimental data measured over a wide frequency range with a Network Analyzer. The six schemes are briefly discussed below.

FIG. 3 illustrates the frequency response 50 of an RF choke 52 connected between an input port 54 and an output port 56. The plot shows that the output signal begins to drop at about 0.5 MHz and continues to drop to −55 dB at about 90 MHz. Above 90 MHz, the output signal climbs back to about 30 db at 150 MHz.

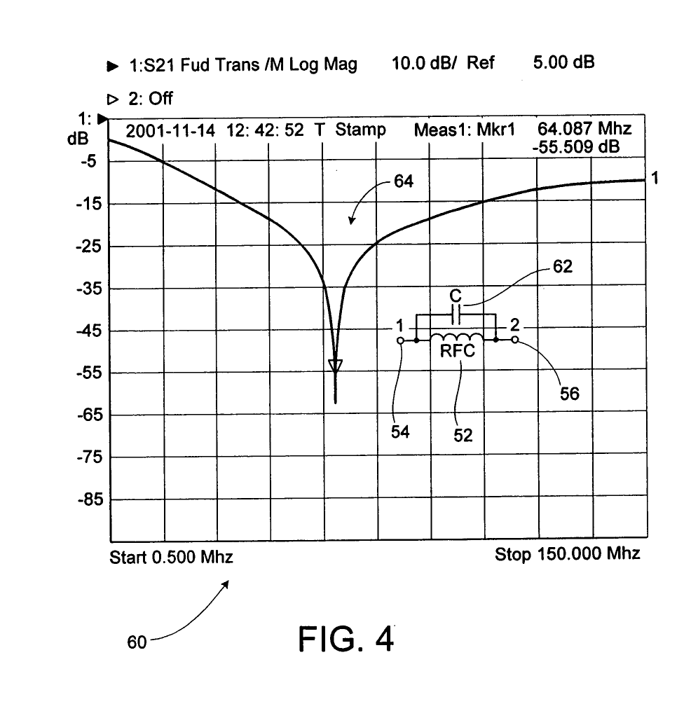

Moving to FIG. 4, a frequency response 60 of another filter network is illustrated. An RF choke 52 and tuning capacitor 62 are connected in parallel, and the parallel combination is connected at one end to an input port 54 and connected on the other end to an output port 56. The frequency response of this configuration is similar in shape to the frequency response 50 shown in FIG. 3. However, the frequency response 60 of FIG. 4 shows a pronounced notch 64 at about 65 MHz, and the output signal climbs sharply back to about −10 dB at 150 MHz.

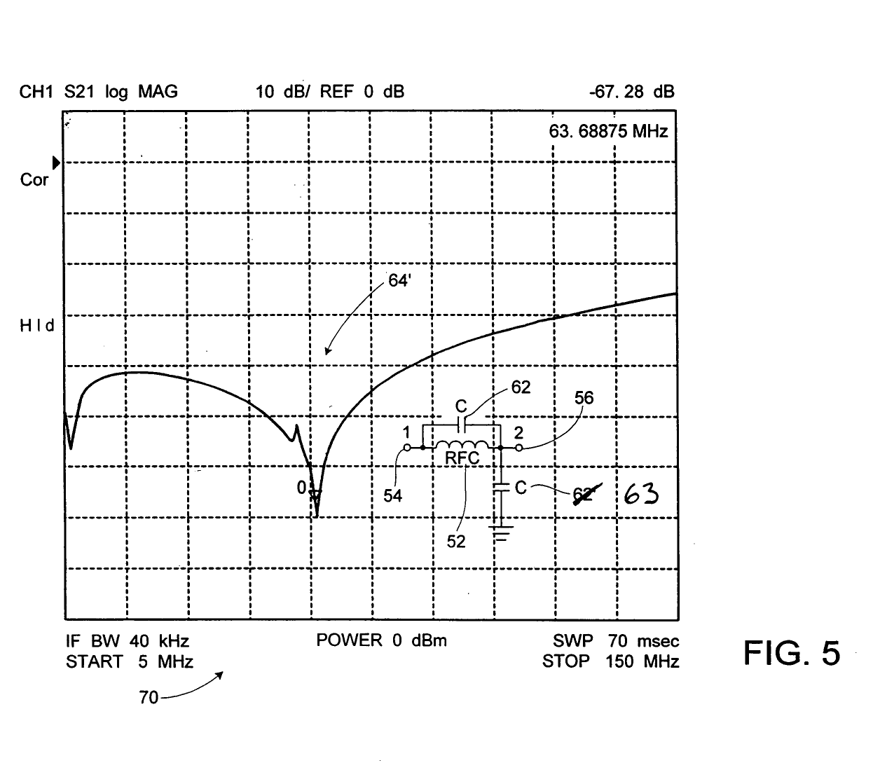

FIG. 5 illustrates the frequency response 70 of another filter network. An RF choke 52 and a tuning capacitor 62 are connected in parallel and the parallel combination is connected at one end to an input port 54 and connected on the other end to an output port 56. An RF shorting capacitor 63 is connected between ground and the output port 56. The frequency response 70 illustrated in FIG. 5 exhibits similar characteristics as the frequency response 60 illustrated in FIG. 4, although the notch 64′ is not as pronounced as the notch 64 of FIG. 4.

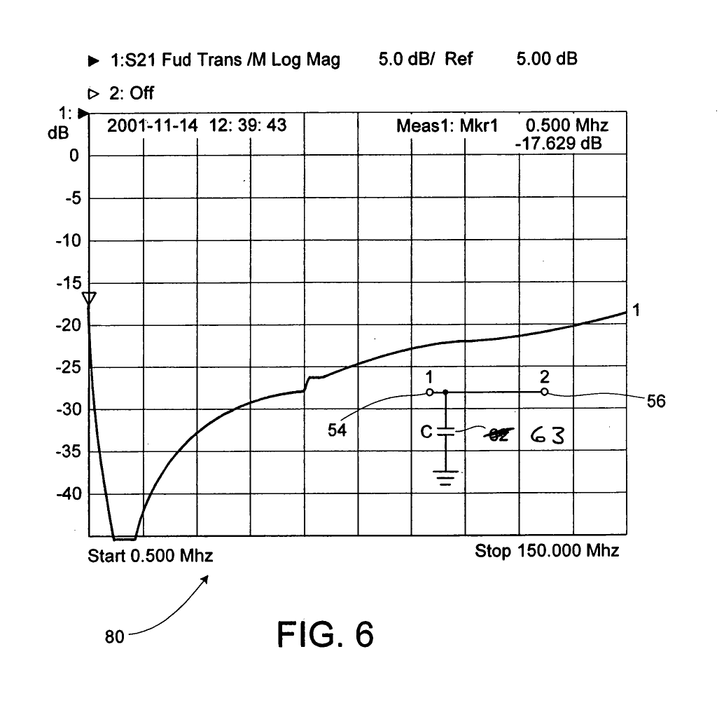

FIG. 6 illustrates the frequency response 80 of an RF shorting capacitor 63 connected between ground and an input port 54, wherein the input port 54 is directly connected to the output port 56. As can be seen in FIG. 6, below 30 MHz the output signal dips severely. Above 30 MHz, the output signal climbs, reaching about −14 db at 150 MHz.

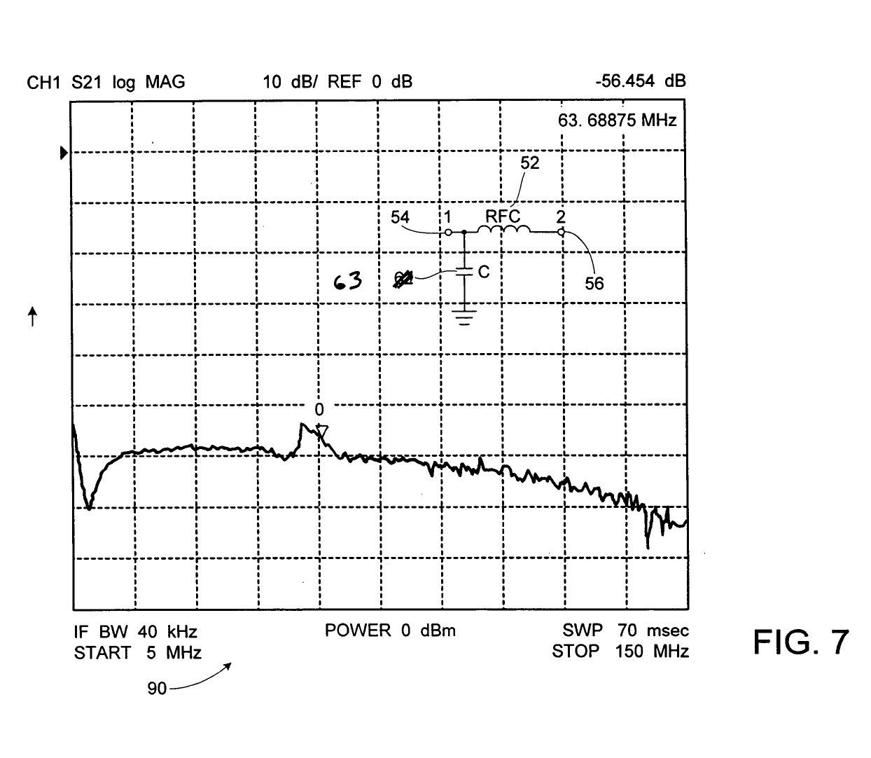

The frequency response 90 of another filter network is shown in FIG. 7. An RF choke 52 is connected between an input port 54 and an output port 56, wherein an RF shorting capacitor 63 is connected between ground and the input port 54. The circuit exhibits a flat response, generally remaining at about −60 dB. Slight variations are seen below 15 MHz and above 120 MHz.

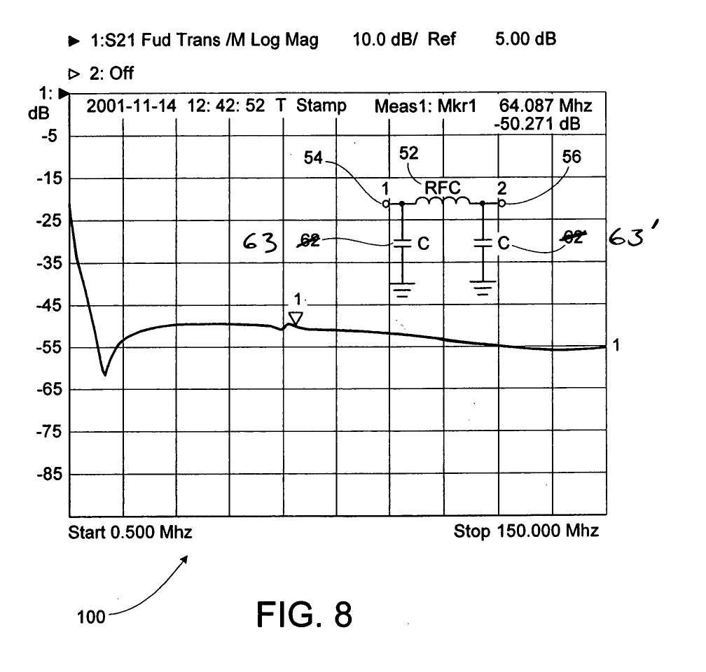

FIG. 8 illustrates the frequency response 100 of an RF choke 52 connected between an input port 54 and an output port 56. A first RF shorting capacitor 63 is connected between ground and the input port 54, and a second RF shorting capacitor 63′ is connected between ground and the output port 56. The circuit exhibits an initial dip below 15 MHz, and levels off to about −50 dB at higher frequencies. Above 105 MHz, the response dips slightly.

From FIGS. 3-8, it can be summarized that if a single frequency of operation is desired, then the circuit of FIG. 4 is preferred (narrow-band). If performance over a wide range is preferred, then the circuit of FIGS. 7 or 8 is preferred (broad-band).

Depending on the need, either series trap block circuits using RF chokes or parallel shunts to the ground via high value RF shorting capacitors can be employed. The value of the RF choke and RF shorting capacitors is chosen based on the frequency of operation for optimum performance. Narrow and broad band filtering can be accomplished in this manner.

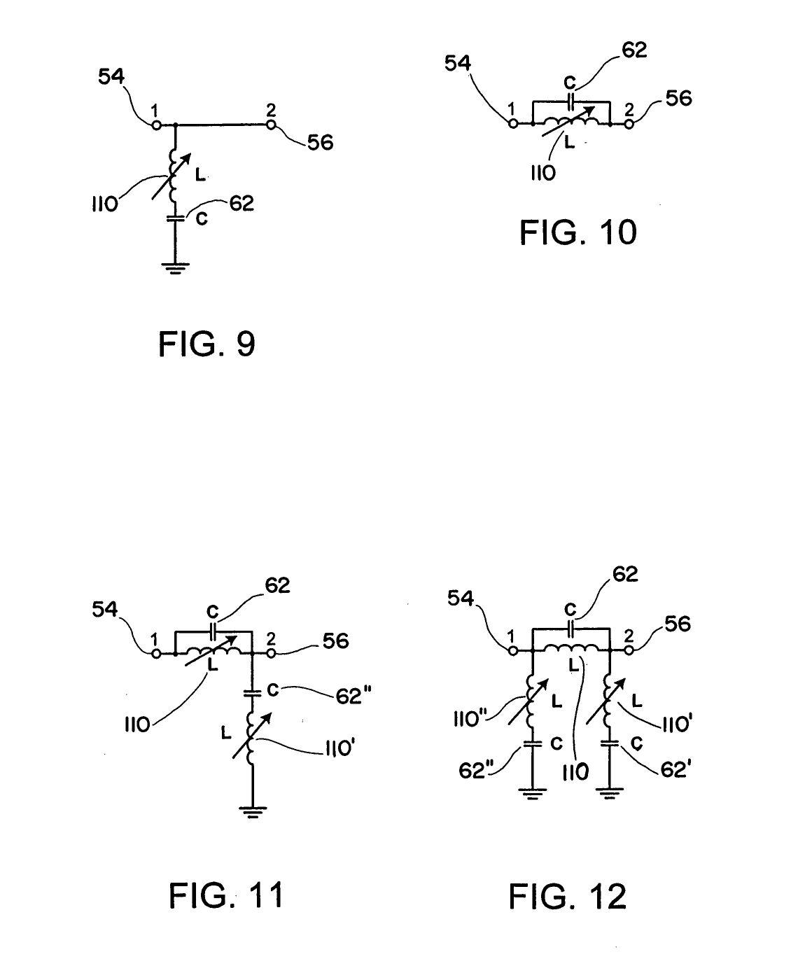

For even greater attenuations of active lines that carry large currents, the circuits of FIGS. 9-12 can be utilized. The circuit of FIG. 9 includes a variable RF choke or inductor 110 connected in series to one end of a suitable tuning capacitor 62. The other end of the tuning capacitor 62 is connected to ground. An input port 54 is connected to the other end of the RF choke 110, and an output port 56 is connected to the input port 54.

The circuit of FIG. 10 includes a variable RF choke 110 connected in parallel with a tuning capacitor 62. One end of the parallel combination is connected to an input port 54, and the other end of the parallel combination is connected to an output port 56.

Moving to FIG. 11, a circuit is shown that includes a first variable RF choke 110 connected in parallel with a tuning capacitor 62. One end of the parallel combination is connected to an input port 54, and the other end of the parallel combination is connected to an output port 56. One end of a second tuning capacitor 62′ is connected to the output port 56, and the other end of the second tuning capacitor is connected to a second variable RF choke 110′. The other end of the second variable RF choke 110′ is connected to ground.

The circuit of FIG. 12 includes a first variable RF choke 110 connected in parallel with a first tuning capacitor 62. One end of the parallel combination is connected to an input port 54, and the other end of the parallel combination is connected to an output port 56. One end of a second variable.RF choke 110′ is connected to the output port 56, and the other end of the second RF choke 110′ is connected to a tuning capacitor 62′. The other end of the second tuning capacitor 62′ is connected to ground. One end of a third variable choke 110″ is connected to the input port 54, and the other end of the third variable RF choke is connected to one end of a third tuning capacitor 62″. The other end of the third tuning capacitor 62″ is connected to ground.

Again the concept of series block or shunt pass circuits can be used. Roughly 25-30 dB of RF attenuation can be achieved with one section (series short or parallel trap). The number of such sections is determined by the amount of interference and the extent of attenuation sought for optimum performance of the two systems (incubator, MR). The characteristic impedances of the circuits of FIGS. 9-12 are chosen to yield maximum attenuation. Values for a 50 ohm 64 MHz series short or parallel trap are L=1245 nH and C=50 pF.

Effective grounding can minimize the RF noise leakage and interferences to both systems. All of the active or passive wires should be routed through 100% shielded cables (coax, triax) wherein all of the shield grounds are shorted to a common ground, e.g., incubator ground, and routed to a magnet room ground through an AC mains plug ground terminal.

EMI Interference

Electro-magnetic interference (EMI) is minimized by shielding the incubator electronics in an RF tight box, e.g., the electronics compartment 40, and grounding all in-coming and out-going (active, passive) signal lines. Further, all lines should be routed within 100% shielded cables to eliminate interference due to electromagnetic fields (E) and magnetic fields (B). The thickness of the electronics compartment 40, for example, can be about 1/16inch to eliminate any interaction with the time varying gradients.

Performance of the MR scanner by way of measuring different parameters (such as frequency, measuring water line width, etc.) was done on a phantom using an RF coil placed inside the incubator. The frequency of the MR remained to within 0.2 ppm (parts per million), but remained steady when the incubator was left running. Ghosting, geometric distortion and eddy currents were nearly identical and under the MR system specifications. Phantom signal-to-noise ratio (S/N) remained within 2-3% with the incubator ON and OFF, which is very close to or under the daily tolerance for quality assurance of the MR scanner.

Careful considerations of material choice including appropriate design considerations (grounding, shielding), passive filtering as described or active filtering for RF, gradients, and electronics placement logistics are important to minimize the interaction of the accessories to MR and vice versa.

While particular embodiments of the invention have been described in detail, it is understood that the invention is not limited correspondingly in scope, but includes all changes, modifications and equivalents coming within the spirit and terms of the claims appended hereto. For example, the methods disclosed herein can be applied to other MR accessories, such as patient monitoring systems, ventilation systems, injectors, syringe pumps, etc which include purely electrical or mechanical, and/or electromechanical and electronic circuits.

What is claimed is:

1. An incubator for use with a magnetic resonance (MR) system, the incubator minimizing an amount of interference generated during an MR scan of the incubator, comprising:
an incubator housing, comprising,
a patient compartment;
an aggregate compartment coupled to the patient compartment;
an electronics compartment coupled to the aggregate compartment;
an incubator frame, wherein the incubator frame and the incubator housing are constructed as a uni-body assembly; and
a means for inhibiting insertion of the electronics compartment of the incubator housing into the MR system.

2. The incubator of claim 1, wherein the inhibiting means comprises at least one elongated member coupled to the incubator frame.

3. The incubator of claim 1, wherein the inhibiting means comprises at least one electrical switch, wherein the electrical switch disables operation of the MR system upon insertion of the electrical compartment into the MR system.

4. The incubator of claim 1, wherein the incubator is constructed from non-conductive components.

5. The incubator of claim 4, wherein the non-conductive components comprise non-metallic components.

6. The incubator of claim 5, wherein the non-metallic components comprise plastic components.

7. The incubator of claim 1, wherein the incubator is constructed from components that are transparent to a magnetic field generated by the MR system.

8. The incubator of claim 1, wherein the incubator is constructed from non-magnetic components.

9. The incubator of claim 8, wherein the non-magnetic components are selected from the group consisting of strontium, phosphor-bronze, beryllium-copper, copper, aluminum, silver and gold.

10. The incubator of claim 1, further comprising:
a metal container secured to the electronics compartment; and
an electric motor secured inside the metal container.

11. The incubator of claim 10, wherein the metal container is secured inside the electronics compartment.

12. The incubator of claim 1, further comprising:
a power supply for providing power to the incubator, the power supply comprising a transformer, said power supply being remotely located relative to the incubator and the MR system.

13. The incubator of claim 12, wherein the power supply is located at least five feet from the incubator and from the MR system.

14. The incubator of claim 1, further comprising at least one filtering means, wherein the at least one filtering means reduces time varying gradient magnetic filed interference.

15. A method for improving the compatibility of a magnetic resonance (MR) accessory for maintaining or monitoring the health of a patient while undergoing magnetic resonance imaging (MRI) with an MR system, comprising at least one of the steps of:
reducing an interference between the accessory and a static magnetic field of the MR system;
reducing an interference between the accessory and a time varying gradient magnetic field of the MR system;
reducing radio frequency (RF) interference between the accessory and the MR system; and
reducing electro-magnetic interference (EMI) between the accessory and the MR system.

16. The method of claim 15, wherein the step of reducing the interference between the accessory and the static magnetic field of the MR system includes the steps of
forming the accessory from non-interference generating components; and
isolating electrical components from the MR system.

17. The method of claim 16, wherein the step of forming the accessory from non-interference generating components includes the step of forming the accessory from non-conductive components.

18. The method of claim 17, wherein the step of forming the accessory from non-conductive components includes the step of forming the accessory from non-metallic components.

19. The method of claim 18, wherein the step of forming the accessory from non-metallic components includes the step of forming the accessory from plastic components.

20. The method of claim 16, wherein the step forming the accessory from non-interference generating components includes the step of forming the accessory from components that are transparent to a magnetic field generated by the MR system.

21. The method of claim 16, wherein the step forming the accessory from non-interference generating components includes the step of forming the accessory from non-magnetic components.

22. The method of claim 21, wherein the step of forming the accessory from non-magnetic components includes the step of forming the accessory from at least one of strontium, phosphor-bronze, beryllium-copper, copper, aluminum, silver and gold.

23. The method of claim 16, wherein the step of forming the accessory from non-magnetic components includes the step of using metallic components that have a low permeability.

24. The method of claim 15, wherein the step of reducing the interference between the accessory and the static magnetic field of the MR system includes the step of shielding electrical components.

25. The method of claim 24, wherein the step of shielding electrical components includes at least one of:
placing electro-magnetic components within a magnetically shielded structure; and
locating electro-magnetic components remotely from the MR system.

26. The method of claim 24, wherein the step of locating the electro-magnetic components remotely from the accessory and the MR system includes the step of locating the electro-magnetic component at least five feet from the MR system.

27. The method of claim 15, wherein the step of reducing the interference between the accessory and the time varying gradient magnetic field of the MR system includes the step of using a filter in at least one signal line.

28. The method of claim 27, wherein the step of using a filter in at least one signal line includes at least one of:
shunting interference signals to ground; and
blocking interference signals from passing through the at least one signal line.

29. The method of claim 15, wherein the step of reducing the interference between the accessory and the time varying gradient magnetic field of the MR system includes at least one of:
minimizing a number of moving metallic parts of the accessory; and
placing metal sections away from a gradient cross-over along a magnet axis.

30. The method of claim 15, wherein the step of reducing RF interference between the accessory and the MR system includes the step of filtering at least one of an active line and a passive line.

31. The method of claim 16, wherein the step of placing a filter in at least one of an active line and a passive line includes the step of using at least one of an active filter and a passive filter to filter the RF interference.

32. The method of claim 30, wherein the step of filtering at least one of an active line and a passive line includes at least one of:
shunting RF signals to ground; and
blocking RF signals from passing through the active line and the passive line.

33. The method of claim 15, wherein the step of reducing EMI between the accessory and the MR system includes the steps of:
shielding at least one electronic component in an RF tight box;
using shielded cable for at least one signal line; and
grounding at least one signal line.

34. The method of claim 15, further comprising the step of using at least one of an incubator, a patient monitor, a ventilator, an injector, a syringe pump as the MR accessory.

35. The method of claim 15, further comprising the step of inhibiting improper insertion of the accessory into the MR system.

36. The method of claim 35, wherein the step of inhibiting improper insertion of the accessory into the MR system includes the step of using at least one of an electrical stop and a mechanical stop.
